(19) United States
(12) Patent Application Publication
(10) Pub. No.: US 2006/0051311 A1
(43) Pub. Date: Mar. 9, 2006

(54) Hair treatment composition containing a combination of at least three different polymers

(76) Inventors: Andrea Walter, Buettelborn (DE); Martina Runge, Mannheim (DE); Susanne Birkel, Darmstadt (DE); Iris Haselbauer, Stockstadt (DE)

Correspondence Address: STRIKER, STRIKER & STENBY, 103 EAST NECK ROAD, HUNTINGTON, NY, 11743, US

(21) Appl. No.: 11/214,962

(22) Filed: Aug. 30, 2005

(30) Foreign Application Priority Data
Sep. 4, 2004 (DE) 10 2004 042 848.4

(51) Int. Cl. A61K 8/81

(52) U.S. Cl. 424/70.16

(57) ABSTRACT

The hair treatment composition contains a combination of an anionic polysaccharide, a second homopolymer or copolymer, which is built up from acrylamidoalkylsulfonic acids, methacrylamidoalkylsulfonic acids, or their salts, and a third polymer, which is an amphiphilic polymer. Preferably the hair treatment composition is a hair styling gel containing xanthan as a first polymer, ammonium acryloyldimethyltaurate/vinylpyrrolidone copolymer as a second polymer and, as a third polymer, cross-linked copolymers made from (meth)acrylic acid and C₁₀- to C₃₀-alkyl esters of (meth)acrylic acid. The preferred gel has a shaky pudding-like consistency and is suitable as a 2-in-1 gel that can be used as either a hair styling gel for increasing hairstyle volume or as a finishing gel for structuring a hairstyle.

### BACKGROUND OF THE INVENTION

The subject matter of the present invention is a hair treatment composition containing a combination of at least three different polymers, and indeed an anionic polysaccharide; a homopolymer or copolymer, which is built up from acryl- or methacrylamidoalkylsulfonic acids or their salts and an amphiphilic polymer. This composition is preferably a hairstyling gel that contains a xanthan, ammonium acryloyidimethyltaurate/vinylpyrrolidone copolymer and a cross-linked copolymer, made from (meth)acrylic acid and a C₁₀- to C₃₀-alkyl esters of (meth)acrylic acid.

Known hairstyling gels can be divided into two groups according to their effectiveness depending on different specific ingredients and thus their different applications connected therewith. First there are the so-called finishing gels, which are applied to dry hair without additional heating action, in order to impart to the hair definition, structure, hold, wet-look and/or luster. Then there are the so-called blow-dry gels, which are applied to moist hair and subsequently the treated hair is dried by heating with a hair drier, to obtain a volume effect in the hairstyle. Usually finishing gels are not suitable or in a position to be able to be applied to moist hair and to provided a volume effect. Similarly conventional blow-dry gels are generally not in a position to be applied to dry hair and to obtain the above-mentioned characteristic actions of a finishing gel.

### SUMMARY OF THE INVENTION

Thus one purpose of the present invention is to provide a 2-in-1 product, which has both a volume-imparting action, when it is applied as a blow-dry gel on moist hair, and also has a structuring effect on the hair or hairstyle, when it is applied to dry hair as a finishing gel.

Furthermore cosmetic products, especially in the rapidly changing and very fashion oriented field of hairstyling products, must be characterized by good application properties and product action and additional properties that are pleasing to the user, e.g. especially new or extraordinary product consistency. There is thus a difficulty that extraordinary consistency, e.g. exceptional rheologic properties, frequently is associated with an inherent reduction in application properties or primary product action.

It is another object of the present invention to provide a hairstyling agent with an attractive consistency differing from conventional styling agents, without impairing the application properties or product performance in an unacceptable manner.

It was found that a 2-in-1 hairstyling agent with a certain combination of three types of polymers can be made and that this hairstyling agent can have an extraordinarily attractive consistency using certain manufacturing processes, without impairing the product performance in an unacceptable manner.

According to the present invention this hair treatment composition contains a combination of

(A) at least one first polymer, which is selected from the group consisting of anionic polysaccharides;

(B) at least one second polymer, which is selected from the group consisting of homopolymers or copolymers, which are built up from at least one type of monomer, which is selected from the group consisting of acryl- or methacrylamidoalkyl sulfonic acids and their salts; and

(C) at least one third polymer different from the at least one first polymer and the at least one second polymer, which is selected from the group consisting of amphiphilic polymers.

The anionic polysaccharide (A) is preferably contained in the hair treatment composition in an amount of from 0.05 to 5 percent by weight or from 0.1 to 1 percent by weight, especially from 0.2 to 0.5 percent by weight. The second polymer (B) is preferably contained in the composition in an amount of 0.1 to 10 percent by weight or from 0.2 to 5 percent by weight, especially from 0.3 to 2 percent by weight. The third polymer (C) is preferably contained in the composition in an amount of 0.01 to 5% by weight or from 0.05 to 2% by weight, especially from 0.1 to 0.5% by weight.

Anionic Polysaccharides (A)

Hydrocolloids based on polysaccharides include polymers built up from saccharide monomers. These polymers can be derivatized, for example by etherization of hydroxyl groups. Anionic polysaccharides are polymers built up from saccharide monomers, which have acid groups, which can be neutralized by suitable neutralizing agents. Nonionic polysaccharides have no acid groups. The polymer (A) can for example be selected from the group consisting of xanthan, gellan, carboxymethyl cellulose, hydroxypropyl cellulose, methyl cellulose, hydroxypropyl-methyl cellulose, hydroxyethyl cellulose, agar-agar, carrageenan, alginate, carob bean meal, guar gum, gum arabic, karaya gum, tragacanth, India gum, pectin and hydroxypropyl guar. Xanthan is a preferred anionic polysaccharide (INCI name: Xanthan gum). Xanthan is a water-soluble natural polysaccharide and contains D-glucose, D-mannose and D-glucuronic acid as individual components and besides that even acetyl and pyruvyl groups. It is a matter of a β(1,4)-glucan chain, in which the 3-position of the glucose molecule is connected with a side chain, which comprises two mannose units and a glucouronic acid unit. The molecular weight MR is preferably in a range from about 10⁵ to 10⁸, especially preferably up to 10⁷.

(Metha)acrylamidoalkylsulfonic Acid Polymer (B)

The second polymer used in the composition of the invention is a homopolymer or copolymer, built up from at least one type of monomer, which is selected from the group consisting of acryl- or methacrylamidoalkylsulfonic acids and their salts. Suitable salts are, e.g., those with ammonium cations, alkali metal cations or alkaline earth metal cations. The polymer is preferably built up from monomers of the general formula H₂C═CH—C(═O)—NH-A-SO₃H or their salts, wherein A is a divalent C₂- to C₆-, preferably C₃- to C₄-, hydrocarbon group. The —C(CH₃)₂—CH₂— group is especially preferred. These monomers are preferably copolymerized with at least one non-ionic, radically copolymerizable monomer, especially a vinyl lactam, especially preferably a vinyl pyrrolidone. A polymer with the INCI name ammonium acryloyldimethyltaurate/VP copolymer is especially preferred. ARISTOFLEX® AVC is especially preferred.

Amphiphilic Polymer (C)

Amphiphilic polymers contain at least one hydrophilic group and at least one hydrophobic group, such as a fat chain. A fat chain can, e.g., be a hydrocarbon group with 8 to 30 or 10 to 22 carbon atoms. Associative thickeners can be used as the amphiphilic polymer. The hydrophobic portions of the molecule can be reduced quantitatively in relation to the remainder of the polymer chain in amphiphilic polymers. They can be distributed statistically along the chain (statistical copolymer), they can be distributed in the form of blocks (block copolymers or sequential polymers) or they can be present as a graft branch or branches (graft copolymers). The amphiphilic polymers can be water-soluble, water-dispersible or they can swell in water. They can be polymers of natural origin, which are modified as needed, or they can be synthetic polymers. Synthetic polymers can, e.g., be made by radical polymerization (e.g. vinyl- or acrylate polymers) or by polycondensation (e.g. polyester or polyurethane). They can also be used as mixtures of the above-mentioned polymers. The amphiphilic polymers can be ionic or nonionic, but anionic and nonionic polymers are preferred.

The amphiphilic polymers (C) are preferably selected from the following:
(1) copolymers of acryl- or methacrylic acid and monomers, which have at least one fat chain; copolymers of this type are, e.g.:
   cross-linked acrylic acid/C₁₀- to C₃₀-alkylacrylate copolymers, e.g. PEMULEN® TR1, PEMULEN® TR2, CARBOPOL® 1382, CARBOPOL® 1342, CARBOPOL® ETD 2020;
   (meth)acrylic acid/ethylacrylate/alkylacrylate copolymers, e.g. ACUSOL® 823, IMPERON R®;
   cross-linked acrylic acid/vinyl isododecanoate copolymers, e.g. STABYLENE(® 30;
   acrylic acid/vinyl pyrrolidone/lauryl methacrylate terpolymers, e.g. ACRYLIDONE LM®, ACP-1184®, ACP-1194®;
   acrylic acid/lauryl(meth)acrylate copolymers, e.g. COATEX SX®;
   (meth)acrylic acid/alkyl acrylate/alkyl(polyethoxy)allyl ether, e.g. RHEOVIS® CR, CR3, CR2 AND CRX;
   methacrylic acid/ethyl acrylate/stearyl(polyethoxy)allyl ether, e.g. SALCARE® SC90 and SC80;
   methacrylic acid/ethyl acrylate/polyethoxylated laurylacrylate terpolymer, e.g. RHEO 2000®;
   methacrylic acid/ethyl acrylate/polyethoxylated stearylmethacrylate terpolymer, e.g. ACRYSOL® 22, ACRYLSOL® 25 and DW-1206A of Rohm & Haas;
   methacrylic acid/polyethoxylated nonylphenol acrylate copolymer, e.g. RHEO 3000®;
   acrylic acid/polyethoxylated stearyl- or cetylmonoitaconate, e.g. STRUCTURE® 2001 AND 3001;
   copolymers of methacrylic acid, butylacrylate and a hydrophobic monomer with at least one fat chain, e.g. the product 8069-146A of National Starch;
   terpolymers of acrylic acid/C₁₅-alkylacrylate/polyethylene glycol acrylate (28 mol ethylene oxide), e.g. DAPRAL GE 202®;
   partial fatty acid ester salts of a copolymer of acrylic acid/dimethylethanol amine, e.g. DAPRAL GE 202 DMA®;
   copolymers of acrylic acid, acrylate and an amphiphilic monomer, which has a fatty chain with urethane groups, e.g. ADDITOL VWX 1312®;
   acryl copolymers modified with fat chains with hydrophobic groups, such as ACUSOL 102®;
(2) Polysaccharides, which are modified with groups, which have at least one fat chain. Copolymers of this type are, e.g.:
   cellulose compounds or their derivatives, which are modified with groups, which have at least one fat chain, e.g. alkyl- , arylalkyl- or alkylaryl groups or their mixtures, wherein the alkyl groups have 8 to 22 carbon atoms;
   nonionic alkylhydroxyethyl celluloses, e.g. NATROSOL® Plus Grade 330 CS, POLYSURF® 67 and ADX 401 (C16-alkyl) from Aqualon;
   quaternary alkylhydroxyalkyl celluloses, e.g. QUATRISOFT®) LM 200; QUATRISOFT® LM-X 529-18-A, QUATRISOFT® LM-X 529-18-B (C12-Alkyl), QUATRISOFT® LM-X 529-8 (C18-Alkyl), CRODACEL® QM, CRODACEL® QL (Cl2-Alkyl), CORDACEL® QS (C18-Alkyl);
   nonionic nonoxynylhydroxyethyl celluloses, e.g. AMERCELL® HM-1500;
   nonionic alkylcelluloses, e.g. BERMOCELL®) EHM 100;
   poly-C₁₂- to C₁₈-alcohol saccharides, e.g. EMULSAN® (D-galactosamine/aminouronic acid mixture), BIOSAN® LPS-50;
   Hydroxyalkyl guar gum modified with a fat chain, e.g. ESAFLOR® HM 22 (modified with C₂₂ alkyl), MIRACARE® XC 95-3 (modified with C₁₄-alkyl), RE 205-146 (modified with C₂₀ alkyl) from Rhone-Poulenc;
(3) Copolymers of maleic acid anhydride or one of its derivatives with monomers, which have at least one fat chain. Copolymers of this type are, e.g.:
   N-octadecylvinyl ether/maleic acid anhydride copolymers, e.g. GANTREZ® AN-8194;
   vinylacetate/isobutylmonomaleate/vinyl neodecanoate terpolymer, e.g. the products ACV4033 and 9649-147 of ISP, MEYPRO-FIX 509®, DENSODRIN®) BA, LIPODERM LIQUOR FP;
(4) Polyurethanes and their derivatives, which carry groups, which have at least one fat chain; e.g. RHEOLATE® 204, 205, 208, 210, 255 and 278; BERMODOL PUR® 2130; ACRYSOL® SCT-275, ACRYSOL® RM-870, ACRYSOL® RM-825, ACRYSOL® 44, ACRYSOL® 46, DW-1206 B, DW-1206 F, SW-1206 G and DW-1206 J of Rohm and Haas; DAPRAL® T 212, SER-AD FX 1100, BORCHIGEL® L.75.N;
(5) Copolymers of crotonic acid and monomers, which have at least one fat chain, e.g. vinyl acetate/crotonic acid/allyl stearate terpolymers;
(6) copolymers of N-vinyl pyrrolidone and monomers, which have at least one fat chain, e.g. olefins substituted with an alkyl group, which contain a long-chain hydrocarbon group, e.g. ANTARON® V216 and ANTARON® V220 of ISP;
(7) nonionic copolymers of acrylic or methacrylic acid alkyl esters with C₁- to C₆-alkyl groups and amphiphilic monomers with fat chains, e.g. copolymers of methylmethacrylate and polyethoxylated stearyl acrylate, e.g. ANTIL® 208;
(8) nonionic copolymers of hydrophilic acrylates or methacrylates and hydrophobic monomers with fat chains, e.g. copolymers of polyethylene glycol methacrylate and alkyl methacrylate.

Preferred Forms and Embodiments

The agent according to the invention is preferably provided in an aqueous, alcoholic or in an aqueous-alcoholic medium with preferably at least 10 percent by weight, especially at least 50% by weight, water and preferably a maximum of 40 percent by weight alcohol. Lower monoalcohols with 1 to 4 carbon atoms, which are usually used for cosmetic purposes, such as ethanol and isopropanol, can be contained in the composition of the invention to provide the alcohol content.

The copolymers of acrylic or methacrylic acid and alkyl esters of acrylic or methacrylic acid, especially cross-linked acrylic acid/C₁₀- to C₃₀-alkylacrylates copolymers, e.g. PEMULEN® TR1, PEMULEN® TR2, CARBOPOL® 1382, CARBOPOL® 1342 and CARBOPOL® ETD 2020, are especially preferred amphiphilic polymers (C).

An especially preferred embodiment of the hair treatment agent according to the invention is a gel containing a combination of

(A) 0.1 to 1 percent by weight xanthan,

(B) 0.2 to 5 percent by weight ammonium acryloyldimethyltaurate/vinyl pyrrolidone copolymer, and

(C) 0.05 to 2 percent by weight of a cross-linked copolymer, made from a first monomer, which is selected from acrylic acid and methacrylic acid, and at least one second monomer, which is selected from C₁₀- to C₃₀-alkyl esters of acrylic or methacrylic acid.

In a preferred embodiment the gel contains polyhydric alcohols (polyols), preferably those with 2 to 6 carbon atoms and with 2 to 6 hydroxy groups, in an amount of 0.1 to 15, preferably of 1 to 10, percent by weight, in order to improve further luster. Glycerol, ethylene glycol, propylene glycol, especially 1,2-propylene glycol and sorbitol, are particularly preferred as the polyols in the composition of the invention.

The polymers containing acid groups are preferably organic or inorganic bases partially or entirely, preferably from 50 to 100%, neutralized. Suitable neutralization agents are primary or secondary amines, especially aminoalkanols with preferably 1 to 10 carbon atoms and 1 to 3 hydroxy groups, such as aminomethylpropanol (AMP), triethanolamine, tetrahydroxypropyl-ethylenediamine or monoethanolamine. However inorganic bases, such as ammonia, NaOH, KOH, among others, may also be used.

The agent according to the invention preferable has a pH in a range from 6to9.

The hair treatment agent according to the invention preferably is present in the form of a gel. The viscosity of the gel according to the invention amounts preferably to from 1000 to 100,000, particularly preferably from 10,000 to 50,000, mpa·s, but most preferably from 25,000 to 35,000 mPa·s, measured as a dynamic viscosity with a HAAKE VT-550 rheometer, measurement body SV-DIN at a temperature of 25° C. and a shear rate of 50 s⁻¹.

The gel can be colored or colorless, It can be clear, transparent or at least translucent, but it can also be non-transparent and turbid, e.g. because it contains pigments, especially pearlescent pigments or other insoluble materials.

Additional Film-Forming or Hair-Fixing Polymer

In additional embodiments the composition according to the invention can contain at least one film-forming and/or hair-fixing polymer. The film-forming or hair-fixing polymer can be nonionic, anionic, cationic, zwitterionic or amphoteric, however it is preferably nonionic or anionic. It can be a synthetic or natural polymer. The term “natural polymer” also includes chemically modified polymers of a natural origin. Those polymers are preferred, which have a sufficient solubility in water, alcohol or water/alcohol mixtures, in order to be present in the composition of the invention in completely dissolved form. The term “hair fixing polymer” means those polymers which, when used in an amount of from 0.01 to 5% in an aqueous, alcoholic or aqueous-alcoholic solution or dispersion, are in a position to provide hair fixing action when the solution or dispersion is applied to the hair. The film-forming or hair-fixing polymers are used in an amount of from 0.01 to 30 or 0.1 to 15, especially preferably from 0.5 to 10, percent by weight.

Suitable synthetic nonionic hair-fixing polymers are homopolymers or copolymers, which are built up from at least one of the following monomers: vinyl pyrrolidone, vinyl caprolactam, vinyl esters, such as vinyl acetate, vinyl alcohols, acrylamide, methacrylamide, alkylacrylamides, dialkylacrylamides, alkylmethacrylamides, dialkylmethacrylamides, dialkylaminoalkylmethacrylamides, dialkylaminoalkylacrylamides, alkylacrylates, alkylmethacrylates, propylene glycol or ethylene glycol, wherein the alkyl groups of these monomers preferably are C₁- to C₇-alkyl groups, especially preferably C₁- to C₃-alkyl groups. For example, homopolymers of vinyl caprolactams, of vinyl pyrrolidone or of N-vinylformamide are suitable. Further suitable synthetic film-forming, non-ionic hair-fixing polymers are, e.g., copolymerizates of vinyl pyrrolidone and vinyl acetate; terpolymers of vinyl pyrrolidone, vinyl acetate and vinyl propionate; terpolymers of vinyl pyrrolidone, vinyl caprolactam and dialkylamino-alkyl(meth)acrylate; terpolymers of vinyl pyrrolidone, vinyl caprolactam and dialkylaminoalkyl(meth)acrylamide; polyacrylamides; polyvinyl alcohols and hair-fixing polyethylene glycol/polypropylene glycol copolymers. Polyvinyl pyrrolidone and polyvinyl pyrrolidone/vinyl acetate copolymers are especially preferred nonionic polymers. Vinyl lactam homopolymer or copolymers are especially preferred nonionic polymers. Suitable vinyl lactams are e.g. vinyl caprolactam and vinyl pyrrolidone. Polyvinyl pyrrolidone, polyvinyl caprolactam and vinyl pyrrolidone/vinyl acetate copolymers are particularly preferred. Preferred commercial products are LUVISKOL® VA 37 AND LUVISKOL® VA 64.

Suitable anionic hair-fixing polymers can be homopolymers or copolymers with monomer units containing acid groups on a synthetic or natural basis, which are copolymerized, as needed, with co-monomers, which contain no acid groups. A sulfuric acid group, a phosphoric acid group or a carboxylic acid group can be used as the acid group. The carboxylic acid group is particularly preferred. The acid groups can be unneutralized, partially or completely neutralized. They are present preferably in a 50 to 100% anionic form or a neutralized form. The above-mentioned neutralizing agents can be used as neutralization agents. Suitable monomers are unsaturated, radically polymerizable compounds, which carry at least one acid group, especially carboxy vinyl monomers. Suitable monomers containing acid groups include e.g. acrylic acid, methacrylic acid, crotonic acid, maleic acid, maleic acid anhydride or their monoesters, aldehydocarboxylic acids or ketocarboxylic acids.

The co-monomers not substituted with acid groups include, e.g., acryl amide, methacrylamide, alkyl- and dialkylacrylamides, alkyl- and dialkylmethacrylamides, alkylacrylates, alkylmethacrylates, vinyl caprolactone, vinyl pyrrolidone, vinyl ester, vinyl alcohol, propylene glycol or ethylene glycol, amine-substituted vinyl monomers, such as dialkylamino-alkylacrylates, dialkylaminoalkyl-methacrylates, monoalkylaminoalkylacrylates and monoalkylaminoalkylmethacrylates, in which the alkyl groups of these monomers are preferably C₁- to C₇-alkyl groups, especially preferably C₁- to C₃-alkyl groups.

Suitable anionic polymers (different from the component (A)) are especially copolymers of acrylic acid or methacrylic acid with monomers selected from the group consisting of acrylic acid or methacrylic acid esters, acrylamide, methacrylamide and vinyl pyrrolidones, homopolymers of crotonic acid and copolymers of crotonic acid with monomers selected from the group consisting of vinyl esters, acrylic acid esters, methacrylic acid esters, acrylamides and methacrylamides. A suitable natural polymer is, for example, shellac.

Preferred anionic polymers are cross-linked or non-cross-linked vinyl acetate/crotonic acid copolymers. Partially esterified copolymers of vinyl methyl ether and maleic acid anhydride are likewise preferred. Further suitable anionic polymers include, e.g. terpolymers of acrylic acid, alkyl acrylate and N-alkylacrylamide, especially terpolymers of acrylic acid, ethyl acrylate and N-t-butylacrylamide or terpolymers of vinyl acetate, crotonate and vinyl alkanoate, especially vinyl acetate/crotonate/vinyl neodecanoate copolymer.

Suitable film-forming amphoteric polymers are polymers, which contain basic or cationic groups, especially primary, secondary, tertiary or quaternary amine groups, besides acid or anionic groups. For example, amphoteric polymers can be copolymers formed from alkylacrylamides (especially octyl acryl amide), alkylaminoalkylmethacrylate (especially t-butylaminoethylmethacrylate) and two or more monomers, namely acrylic acid, methacrylic acid or their C₁- to C₄-alkylesters. At least one type of monomer contains an acid group, such as the monomers which are marketed under the trademark AMPHOMER® or AMPHOMER® LV-71, of National Starch, USA.

Further suitable hair-fixing polymers are copolymers of acrylic acid, methacrylate and methacrylamidopropyltrimethylammonium chloride (INCI: polyquaternium47), copolymers made from acrylamidopropyltrimonium chloride and acrylates or copolymers made from acrylamide, acrylamidopropyltrimonium chloride, 2-amidopropylacrylamide sulfonate and dimethylaminopropylamine (INCI: Polyquaternium43) or chitosans. Polymers with monomers carrying betaine groups are also suitable, such as copolymers of methacryloylethyl betaine and two or more monomers of acrylic acid or its simple esters, INCI name: Methacryloyl ethyl betaine/acrylates copolymer.

Cationic polymers are polymers with cationic or cationic groups, especially primary, secondary, tertiary or quaternary amine groups. The cationic charge density amounts preferably to 1 to 7 meq/g. Suitable cationic polymers are hair-fixing or hair-conditioning polymers. Suitable polymers preferably contain quaternary amine groups. The cationic polymers can be homopolymer or copolymers, in which the quaternary nitrogen groups are contained either in the polymer chain or preferably as substituents of one or more monomers. The monomers containing ammonium groups can be copolymers with monomers, which are not cationic monomers. Suitable cationic monomers are unsaturated, radically polymerizable compounds, which carry at least one cationic group, especially ammonium-substituted vinyl monomers, e.g. such as trialkylmethacryloxyalkyl ammonium groups, trialkylacryloxyalkyl ammonium groups, dialkyldiallyammonium groups and quaternary vinyl ammonium monomer groups with cyclic, cationic nitrogen-containing groups, such as pyridinium, imidazolium or quaternary pyrrolidones, e.g. alkylvinylimidizolium, alkylvinylpyridinium or alkylvinylpyrrolidone salts. The alkyl groups of these monomers are preferably lower alkyl groups, such as alkyl groups having one to seven carbon atoms, especially preferably from one to three carbon atoms.

The monomers containing ammonium groups can be copolymerized with non-cationic monomers. Suitable comonomers are for example acryl amide, methacrylamide, alkyl- and dialkylacrylamide, alkyl- and dialkylmethacryl amide, alkyl acrylate, alkyl methacrylate, vinyl caprolactone, vinyl caprolactam, vinyl pyrrolidone, vinyl ester, e.g. vinyl acetate, vinyl alcohol, propylene glycol or ethylene glycol, wherein the alkyl groups of these monomers preferably have one to seven carbon atoms, especially one to three carbon atoms.

Suitable polymers with quaternary amine groups are, for example, polymers described in the CTFA Cosmetic Ingredient Dictionary under the name “polyquaternium”, such as methylvinylimidazolium chloride/vinyl pyrrolidone copolymer (Polyquaternium-16) or quaternized vinyl pyrrolidone/dimethylaminoethylmethacrylate copolymer (Polyquaternium-11) and quaternary silicone polymers and/or oligomers, such as silicone polymers with quaternary terminal groups (Quaternium-80).

Preferred cationic polymers on a synthetic basis include: poly(dimethyldiallylammonium chloride); copolymers of acrylamide and dimethyldiallyammonium chloride; quaternary ammonium polymers, formed by reaction of diethyl sulfate and a copolymer of vinyl pyrrolidone and dimethylaminoethylmethacrylate, especially vinyl pyrrolidone/dimethylaminoethylmethacrylate methosulfate copolymer, (e.g. GAFQUAT® 755 N and GAFQUAT® 734); quaternary ammonium polymers of vinyl pyrrolidone and methylvinylimidazolium chloride (e.g. LUVIQUAT HM 550); Polyquaternium-35; Polyquaternium-57; polymers from trimethylammonium-ethyl-methacrylate chloride; terpolymers of dimethyldiallylammonium chloride, sodium acrylate and acrylamide (MERQUAT Plus 3300); terpolymers of vinyl pyrrolidone, dimethylaminopropylmethacrylamide and methacryloylaminopropyllauryldimethyl ammonium chloride; terpolymers of vinyl pyrrolidone, dimethylaminoethylmethacrylate and vinyl caprolactam (e.g. GAFFIX® VC 713); vinyl pyrrolidone/methacrylamidopropyltrimethyl ammonium chloride copolymer (GAFQUAT® HS 100); copolymers of vinyl pyrrolidone and dimethylaminoethylmethacrylate; copolymers of vinyl pyrrolidone, vinyl caprolactam and dimethylaminopropyl acrylamide; polyesters or oligoesters built up from at least one first monomer type, which is a hydroxy acid substituted with at least one quaternary ammonium group; and substituted polydimethylsiloxanes with terminal quaternary ammonium groups.

Suitable cationic polymers, which are derived from natural polymers, are especially cationic derivatives of polysaccharides, e.g. cellulose, starch or guar. Chitosan and chitosan derivative compounds are suitable. Cationic polysaccharides have the general formula (I):
G-O—B—N⁺R^{a}R^{b}R^{c}X⁽⁻⁾ (I),
wherein G is an anhydroglucose residue, for example starch or cellulose anhydroglucose; B is a divalent connecting group, for example, an alkylene, an oxyalkylene, a polyoxyalkylene or hydroxyalkylene;
R^{a}, R^{b} and R^{c} are each, independently of each other, alkyl, aryl, alkylaryl, arylalkyl, alkoxyalkyl or alkoxyaryl with up to 18 carbon atoms respectively, wherein the total number of carbon atoms in R^{a}, R^{b} and R^{c} is at most 20; X is a common counter anion, for example a halogen, acetate, phosphate, nitrate or alkyl sulfate, preferably chloride. Preferred cationic celluloses are those marketed under the INCI name, polyquaternium-10 and polyquaternium-24. A suitable cationic guar derivative is e.g. the derivative with the INCI name, guar hydroxypropyltrimonium chloride.

Chitosan, chitosan salts and chitosan derivative compounds are especially preferred as cation-active materials. The chitosan used in the composition of the invention is partially or completely deacetylated. The molecular weights of chitosan can vary over a wide range, for example from 20,000 to 5,000,000 g/mol. A low molecular weight chitosan is, for example, considered to be a chitosan with a molecular weight of from 30,000 to 70,000 g/mol. Preferably the molecular weight of the chitosan is above 100,000 g/mol, especially preferably form 200,000 to 700,000 g/mol. The deacetylation degree amounts to from 10 to 99%, especially preferably from 60 to 99%. A preferred chitosan salt is chitosonium pyrrolidone carboxylate, which, for example, is marketed under the trade name KYTAMER® PC of Amerchol, USA. The chitosan obtained has a molecular weight of about 200,000 to 300,000 g/mol and is deacetylated up to 70 to 85%. Quaternary, alkylated or hydroxyalkylated derivative chitosan compounds, for example, the hydroxyethyl chitosan, hydroxypropyl chitosan or hydroxybutyl chitosan, are suitable in the compositions according to the invention. The chitosans or chitosan derivative compounds should be present in neutralized or partially neutralized form when used in the compositions of the invention. The neutralization degree for the chitosan or the chitosan derivative compounds is preferably at least 50%, especially preferably between 70 and 100%, relative to the number of free base groups. In principle, all cosmetically compatible inorganic or organic acids may be used as neutralization agent, for example formic acid, tartaric acid, malic acid, lactic acid, citric acid, pyrrolidone carboxylic acid, hydrochloric acid, among other. Pyrrolidone carboxylic acid is especially preferred as neutralization agent.

Preferred cationic polymers on a natural basis include: cationic cellulose derivatives comprising hydroxyethyl cellulose and diallyldimethyl ammonium chloride; cationic cellulose derivatives comprising hydroxyethyl cellulose and with trimethyl ammonium-substituted epoxides; chitosan and its salts; hydroxyalkyl chitosans and their salts; alkylhydroxyalkyl chitosans and their salts and N-hydroxyalkyl chitosan alkyl ether.

Temporary Hair Coloring Ingredients

In a preferred embodiment of the invention the composition according to the invention includes at least one temporary hair coloring pigment for simultaneously fixing and temporary hair coloring. The term “temporary hair coloring” means a color change of human hair, which is maintained until the next hair washing and can be removed again by washing the hair with conventional shampoos. The pigments are preferably contained in an amount of 0.01 to 25 percent by weight, especially preferably from 5 to 15 percent by weight. The pigments are preferably micro-pigments, not nano-pigments. The preferred particle size amounts to from 1 to 200 μm, especially from 3 to 150 μm, especially preferably from 10 to 100 μm.

The pigments are practically insoluble coloring agents and can be inorganic or organic. Also inorganic-organic mixed pigments may be used. Inorganic pigments are preferable. The advantage of the inorganic pigments is their outstanding light-resistance, weather-resistance and temperature-resistance. The inorganic pigments can be of natural origin, for example chalk, ocher, umber, green earth, burnt sienna or graphite. The pigments can be white pigments, such as titanium dioxide or zinc oxide; black pigments, such as iron oxide black; fancy or multi-colored pigments, such as ultramarine or iron oxide red; lustrous pigments, metal effect pigments, pearlescent pigments as well as fluorescent or phosphorescent pigments. Preferably at least one pigment is a colored, non-white pigment. Metal oxides, metal hydroxides and metal oxide hydrates, mixed phase pigments, sulfur-containing silicates, metal sulfides, complex metallo-cyanides, metal sulfates, metal chromates and metal molybdates and metals themselves (bronze pigments) are suitable. In particular, titanium dioxide (C.I. 77891), black iron oxide (C.I. 77499), yellow iron oxide (C.I. 77492), red and brown iron oxide (C.I. 77491), manganese violet (C.I. 77742), ultramarine (sodium aluminum sulfo-silicate, C.I. 77007, Pigment Blue 29), chromium oxide hydrate (C.I. 77289), Iron Blue (Ferric ferrocyanide, C.I. 77510) and carmine (cochineal), are all suitable pigments. Pigments based on mica and/or isinglass, which are coated with a metal oxide or metal oxychloride, such as titanium dioxide or bismuth oxychloride and if necessary other color-imparting materials, such as iron oxides, iron blue, ultramarine, carmine, etc, whose colors can be modified by changing the thickness of the coating, are especially preferred. Pigments of this sort are marketed, for example, under the trademark, Rona®, Colorona®, Dichrona® and Timiron® by the firm, Merck, Germany. Organic pigments are, for example, the natural pigments, Sepia, gamboge, charcoal, Kasseler brown, indigo, chlorophyl and other plant pigments. Synthetic organic pigments include, for example, azo pigments, anthraquinoid pigments, indigoid pigments, dioxazine, quinacridone, phthalocyanine isoindolinone pigments, perylene pigments, perinone pigments, metal complex pigments, alkali blue pigments and diketopyrrolopyrrole pigments.

Fat Ingredient

In additional embodiments of the invention the hair treatment composition contains at least one fat ingredient. The fat ingredient can be present in an amount of e.g. 0.05 to 15% by weight. Fat ingredients are hydrophobic, substantially water-insoluble materials and can, e.g., be:
silicone compounds, which are selected from the group consisting of silicone waxes, silicone oils, cyclic dimethyl siloxanes, linear polydimethyl siloxanes, block polymers made from polydimethyl siloxanes and polyethylene oxide and/or polypropylene oxide, polydimethyl siloxanes with terminal and side polyethylene oxide or polypropylene oxide groups, polydimethylsiloxanes with terminal hydroxyl groups, phenyl substituted polydimethyl siloxanes, silicone emulsions, silicone elastomers, silicone gums and amino-substituted silicones; and
oils and waxes, which are selected from the group consisting of paraffin waxes, polyolefin waxes, wool wax, wool alcohol, candelilla wax, olive wax, carnauba wax, Japan wax, apple wax, hardened fats, fatty acid esters, fatty acid glycerides, fatty acid triglycerides, polyethylene glycol waxes, mineral oils, isoparaffin oils, paraffin oils, squalane, sunflower seed oil, coconut oil, castor oil, lanolin oil, jojoba oil, corn oil and soybean oil.

Conventional Cosmetic Additive Ingredients

The composition according to the invention can also contain conventional additive ingredients that are usually used in hair treatment compositions, for example wetting agents or emulsifies from the classes of nonionic, anionic, cationic or amphoteric surface-active ingredients, such as ethoxylated fatty alcohols, fatty alcohol sulfates, alkyl benzene sulfonates, alkyl trimethyl ammonium salts, alkyl betaines, in an amount of from 0.1 to 15% by weight; moisturizing agents; perfume oils, in an amount of 0.01 to 1 percent by weight; turbidity-inducing agents, such as ethylene glycol distearate, in an amount of about 0.2 to 5.0 percent by weight; pearlescent agents, such as a mixture of fatty acid monoalkylolamide and ethylene glycol distearate, in an amount of about 1.0 to 10 percent by weight, bactericide and fungicide active ingredients, such as 2,4,4-trichloro-2-hydroxydiphenyl ether or methyl chloroisothiazolinone, in an amount of 0.01 to 1.0 percent by weight; buffer substances, such as e.g. sodium citrate or sodium phosphate, in an amount of 0.1 to 1.0 percent by weight; coloring substances, such as fluorescein sodium salt, in an amount of about 0.1 to 1.0 percent by weight; care materials, such as e.g. betaine, panthenol, plant extracts, vegetable extracts, protein hydrolyzates, silk hydrolyzates, lanolin derivatives, in an amount of 0.1 to 5 percent by weight; light-protective agents, antioxidants, radical-trapping agents, anti-flaking agents, in an amount of about 0.01 to 4 percent by weight; fatty alcohols, consistency formers, luster-imparting agents, vitamins, softeners, combability improving agents, de-fatting agents and anti-foaming agents.

Hair Treatment Gel

The subject matter of the present invention also includes a method for making a hair treatment gel and the gel made by this method. The method for making the hair treatment gel includes first making a gel mass based of an aqueous solvent containing
(A) at least one first polymer selected from hydrocolloids based on nonionic polysaccharides or anionic polysaccharides;
(B) at least one second polymer, which is a homopolymer or copolymer, which is selected from the group consisting of acrylamidoalkylsulfonic acids, salts of acrylamidoalkylsulfonic acids, methacrylamidoalkylsulfonic acids and salts of methacrylamidoalkylsulfonic acids; and
(C) at least one third polymer different from the first polymer and second polymer, selected from amphiphilic polymers.

Subsequently the gel mass is stored at temperatures above room temperature (25° C.). A new consistency results from keeping the gel mass at temperatures above room temperature. The storing takes place ideally until the new consistency has completely or nearly completely adjusted and no further changes are observable. The storage temperatures are, e.g., from 30 to 80° C., preferably from 40 to 70° C. or 45 to 60° C. Storage times are preferably at least two hours and at most three days, e.g. five hours to 1.5 days, especially from 7 to 12 h. The storage occurs at elevated temperatures, preferably until a gel-like solidification has taken place. The consistency adjusted during the gel formation can be described as gel-like, gelatinous or shaky pudding-like solid or semi-solid. The end product is then gelatinous, transparent, translucent and/or opaque with a non-uniform pleasing appearance, especially a lumpy consistency with macroscopically observable agglomerates. However it can still be applied by brushing and spreads out sufficiently.

The composition according to the invention can, in principle, be used to treat hair in two different ways. The first use is that of a so-called finish gel. For this purpose as a final (or single) step for hairstyling the composition is applied to dry hair and worked into the hair without application of heat. In this step the entire hair or only a single side or strand can be treated. The treated hair has definition, structure, hold, wet-look and/or luster. A second use is that of a so-called styling gel. The composition is applied to moist hair, e.g. after applying washing the hair or moistening the hair with water. The composition is applied to the moist hair and worked into the hair. The entire hair or only a side or strand of the hair can be treated. Subsequently the treated hair is treated while heating it with a hair drier. A volume-imparting action can be attained in this way. The subject matter of the invention thus includes the use of a hair treatment agent, as described above, as a finishing gel for structuring a hairstyle or hair-do and/or as a styling gel for increasing hair volume of the hairstyle.

The hair treatment composition according to the invention has the following advantages:

Attractive and exceptional consistency (similar to a shaky pudding); good distributability; economical during application; usable as a 2-in-1 gel, i.e. both as a finishing gel and a styling gel; good definition and texture for treatment of hair; good volume action; slight load; great strength; no residue; hair shaped satisfactorily; does not dry too quickly; luster-imparting; suitable for treatment of curly hair, especially for use on moist hair and air drying; suitable for treatment of short hair and setting up a hairstyle.

The following examples should serve to illustrate the subject matter of the invention in greater detail.

### EXAMPLES

The following exemplary formulations of the hair treatment composition of the invention are usable both on moist hair as well as dry hair, i.e. as TWO-IN-ONE gels. These gels have a semi-solid, but are not resistant to cutting, and have a soft, shaky pudding-like consistency and are easily rubbed into the hair after storage over night at about 50° C. In case a higher or lower viscosity is desired, the viscosity can be adjusted by variation of the content of PEMULEN® (Acrylates/C10-30 alkyl acrylate cross-polymer).

### Example 1

### Hair Gel

| | |
|---|---|
| 0.3 | Xanthan gum |
| 1.0 | Ammonium acryloyldimethyltaurate/VP copolymer |
| 0.1 | Acrylates/C10-30 Alkyl Acrylate Crosspolymer |
| 0.08 | AMP-95 |
| 1.5 | PVP |
| 2 | Polyquaternium-11 |
| 5 | Glycerol |
| 0.3 | Perfume |
| 0.3 | EMULGIN ® L (PPG-1-PEG-9 Lauryl Glycol Ether) |
| 1.0 | Dye solution |
| To 100 | Water |

### Example 2

### Hair Gel

| | |
|---|---|
| 0.3 | Xanthan gum |
| 1.0 | Ammonium acryloyldimethyltaurate/VP copolymer |
| 0.2 | Acrylates/C10-30 Alkyl Acrylate Crosspolymer |
| 0.16 | AMP-95 |
| 1.5 | PVP |
| 2 | Polyquaternium-11 |
| 5 | Propylene glycol |
| 0.3 | Perfume |
| 0.3 | CREMOPHOR ® CO 410 (PEG-40 Hydrogenated |
| | Castor Oil) |
| 0.5 | Dye solution |
| To 100 | Water |

### Example 3

### Hair Gel

| | |
|---|---|
| 0.3 | Xanthan gum |
| 1.0 | Ammonium acryloyldimethyltaurate/VP copolymer |
| 0.3 | Acrylates/C10-30 Alkyl Acrylate Crosspolymer |
| 0.23 | AMP-95 |
| 1.5 | PVP |
| 2 | Polyquaternium-11 |
| 5 | Glycerol |
| 0.3 | Perfume |
| 0.3 | CREMOPHOR ® CO 410 (PEG-40 Hydrogenated |
| | Castor Oil) |
| 0.7 | Dye solution |
| To 100 | Water |

### Example 4

### Hair Gel

| | |
|---|---|
| 0.2 | Xanthan gum |
| 1.2 | Ammonium acryloyldimethyltaurate/VP copolymer |
| 0.3 | Acrylates/C10-30 Alkyl Acrylate Crosspolymer |
| 0.23 | AMP-95 |
| 2 | PVP |
| 2.5 | VP/VA Copolymer |
| 5 | Glycerol |
| 0.3 | Perfume |
| 0.3 | CREMOPHOR ® CO 410 (PEG-40 Hydrogenated |
| | Castor Oil) |
| 0.3 | Dye solution |
| To 100 | Water |

### Example 5

### Hair Gel

| | |
|---|---|
| 0.5 | Xanthan gum |
| 0.9 | Ammonium acryloyldimethyltaurate/VP copolymer |
| 0.2 | Acrylates/C10-30 Alkyl Acrylate Crosspolymer |
| 1.0 | AMP-95 |
| 1.5 | VA/Crotonates copolymer |
| 6 | Propylene glycol |
| 0.3 | Perfume |
| 0.3 | EMULGIN ® L (PPG-1-PEG-9 Lauryl Glycol Ether) |
| 1.0 | Dye solution |
| To 100 | Water |

### Example 6

### Hair Gel

| | |
|---|---|
| 0.3 | Xanthan gum |
| 3 | Ammonium acryloyldimethyltaurate/VP copolymer |
| 0.1 | Acrylates/C10-30 Alkyl Acrylate Crosspolymer |
| 0.08 | AMP-95 |
| 4 | VP/methacrylamide/vinyl imidazole copolymer |
| 4 | Paraffinum Perliquidum |
| 0.3 | Perfume |
| 0.3 | CREMOPHOR ® CO 410 (PEG-40 Hydrogenated |
| | Castor Oil) |
| 0.7 | Dye solution |
| To 100 | Water |

### Example 7

### Hair Gel

| | |
|---|---|
| 0.5 | Xanthan gum |
| 2 | Ammonium acryloyldimethyltaurate/VP copolymer |
| 0.5 | Acrylates/C10-30 Alkyl Acrylate Crosspolymer |
| 0.4 | AMP-95 |
| 4 | Polyvinyl pyrrolidone |
| 4 | Glycerol |
| 0.3 | Perfume |
| 0.3 | CREMOPHOR ® CO 410 (PEG-40 Hydrogenated |
| | Castor Oil) |
| 0.6 | Dye solution |
| To 100 | Water |

The disclosure in German Patent Application 10 2004 042 848.4 of Sep. 4, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a hair treatment composition containing a polymer combination, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A hair treatment composition containing a combination of
at least one first polymer consisting of at least one hydrocolloid, said at least one hydrocolloid comprising at least one nonionic or anionic polysaccharide;
at least one second polymer selected from the group consisting of homopolymers and copolymers, each of said homopolymers and copolymers being built up from at least one monomer, wherein said at least one monomer is selected from the group consisting of acrylamidoalkyl sulfonic acids, salts of said acrylamidoalkyl sulfonic acids, methacrylamidoalkyl sulfonic acids and salts of said methacrylamidoalkyl sulfonic acids; and
at least one third polymer different from the at least one first polymer and different from the at least one second polymer, said at least one third polymer consisting of at least one amphiphilic polymer.

2. The hair treatment composition as defined in claim 1, containing from 0.05 to 5 percent by weight of said at least one first polymer, from 0.1 to 10 percent by weight of said at least one second polymer and/or from 0.01 to 5 percent by weight of said at least one third polymer.

3. The hair treatment composition as defined in claim 1, in the form of gel with a viscosity of at least 1000 mPa s, measured as dynamic viscosity with HAAKE VT-550 rheometer, measurement body SV-DIN at a temperature of 25° C. and a shear rate of 50 s-⁻¹.

4. The hair treatment composition as defined in claim 1, wherein said at least one first polymer is selected from the group consisting of xanthan, gellan, carboxymethyl cellulose, hydroxypropyl cellulose, methyl cellulose, hydroxylpropylmethyl cellulose, hydroxyethyl cellulose, agar-agar, carrageenan, alginate, carob bean meal, guar gum, gum arabic, karaya gum, tragacanth, India gum, pectin and hydroxypropyl guar.

5. The hair treatment composition as defined in claim 1, wherein said at least one first polymer is xanthan.

6. The hair treatment composition as defined in claim 1, wherein said at least one monomer has a general formula H₂C═CH—C(═O)—NH-A-SO₃H or is a salt thereof, wherein A is a divalent C₂- to C₆- hydrocarbon group.

7. The hair treatment composition as defined in claim 6, wherein said at least one second polymer is ammonium acryloyldimethyltaurate/vinyl pyrrolidone copolymer.

8. The hair treatment composition as defined in claim 1, wherein the at least one third polymer is a copolymer of acryl- or methacrylic acid and a monomer with at least one fat chain; a polysaccharide, which is modified with at least one group, said at least one group having at least one fat chain; a copolymer of maleic acid anhydride or a derivative thereof with a monomeric compound, said monomeric compound having at least one fat chain; a polyurethane or a derivative thereof, which carries at least one group with at least one fat chain; a copolymer of crotonic acid and a monomeric compound with at least one fat chain; a copolymer of N-vinyl pyrrolidone and a monomer with at least one fat chain; a nonionic copolymer of an acrylic or methacrylic acid alkyl ester with C₁- to C₆-alkyl groups and an amphiphilic monomer with a fat chain; and a nonionic copolymer of a hydrophilic acrylate or methacrylate and a hydrophobic monomer with a fat chain.

9. The hair treatment composition as defined in claim 1, wherein the at least one third polymer is a cross-linked or non-cross-linked copolymer built up from at least one monomer compound and at least one other monomer compound, wherein said at least one monomer compound is selected from the group consisting of acrylic acid and methacrylic acid and at least one other monomer compound is selected from the group consisting of C₈- to C₃₀-alkyl esters of acrylic acid and C₈- to C₃₀-alkyl esters of methacrylic acid.

10. A hair treatment composition containing
from 0.1 to 1 percent by weight of xanthan;
from 0.2 to 5 percent by weight of ammonium acryloyldimethyltaurate/vinyl pyrrolidone copolymer; and
from 0.05 to 2 percent by weight of a cross-linked copolymer comprising at least one first monomer compound and at least one second monomer compound, wherein said at least one first monomer compound is selected from the group consisting of acrylic acid and methacrylic acid and at least one second monomer compound is selected from the group consisting of C₁₀- to C₃₀-alkyl esters of acrylic acid and C₁₀- to C₃₀-alkyl esters of methacrylic acid.

11. The hair treatment composition as defined in claim 10, further comprising at least one film-forming or hair-fixing polymer.

12. The hair treatment composition as defined in claim 10, further comprising at least one fat ingredient.

13. The hair treatment composition as defined in claim 1, further comprising at least one film-forming or hair-fixing polymer.

14. The hair treatment composition as defined in claim 13, wherein said at least one film-forming or hair-fixing polymer is a terpolymer of acrylic acid, ethyl acrylate and N-tert-butylacrylamide; a cross-linked or non-cross-linked vinyl acetate/crotonic acid copolymer; a terpolymer of tert.-butyl acrylate, ethyl acrylate and methacrylic acid; a sodium polystyrene sulfonate; a copolymer of vinyl acetate, crotonic acid and vinyl propionate; a copolymer of vinyl acetate, crotonic acid and vinyl neodecanoate; an aminomethylpropanol-acrylate copolymer; a copolymer of vinyl pyrrolidone and at least one additional monomer, said at least one additional monomer being selected from the group consisting of acrylic acid, methacrylic acid, acrylic acid esters and methacrylic acid esters; a copolymer of methyl vinyl ether and a maleic acid monoalkyl ester; an aminomethylpropanol salt o a copolymer of allyl methacrylate and at least one other monomer, said at least one other monomer being selected from the group consisting of acrylic acid, methacrylic acid, acrylic acid esters and methacrylic acid esters; a copolymer of vinyl acetate, mono-n-butyl maleate and isobornyl acrylate; a copolymer of two or more monomeric compounds, said two or more monomeric compounds being selected from the group consisting of acrylic acid, methacrylic acid, acrylic acid esters and methacrylic acid esters; a copolymer of octylacrylamide and at least one monomer compound, said at least one monomer compound being selected from the group consisting of acrylic acid, methacrylic acid, acrylic acid esters and methacrylic acid esters; a polyester of diglycol, cyclohexandimethanol, isophthalic acid and sulfoisophthalic acid; a copolymer of vinyl caprolactam, vinyl pyrrolidone and dimethylaminoethylmethacrylate; a copolymer of vinyl pyrrolidone and dimethylaminoethylmethacrylate; a copolymer of vinyl pyrrolidone, vinyl caprolactam and dimethylaminopropyl acrylamide; a copolymer of octylacrylamide, acrylic acid, butylaminoethylmethacrylate, methylmethacrylate and hydroxypropylmethacrylate; a copolymer of lauryl acrylate and stearyl acrylate; polyvinyl pyrrolidone; polyvinyl caprolactam; a vinyl pyrrolidone/vinyl acetate copolymer; polyvinyl alcohol; a isobutylene/ethylmaleimide/hydroxyethylmaleimide copolymer; a copolymer of vinyl pyrrolidone, vinyl acetate and vinyl propionate; a copolymer of vinyl pyrrolidone, methacrylamide and vinyl imidazole and/or hydrolyzed corn starch.

15. The hair treatment composition as defined in claim 1, further comprising at least one fat ingredient.

16. The hair treatment composition as defined in claim 15, wherein said at least one fat ingredient comprises at least one silicone compound and/or at least one of oils and waxes;
wherein said at least one silicone compound is selected from the group consisting of silicone waxes, silicone oils, cyclic dimethylsiloxanes, linear polydimethylsiloxanes, block polymers of polydimethylsiloxane and polyethylene oxide and/or polypropylene oxide, polydimethylsiloxanes with terminal or side polyethylene oxide or polypropylene oxide groups, phenyl-substituted polydimethylsiloxanes, silicone emulsions, silicone elastomers, silicone gums and amino-substituted silicones; and
wherein said oils and waxes are selected from the group consisting of paraffin waxes, polyolefin waxes, wool wax, wool alcohol, candelilla wax, olive wax, carnauba wax, Japan wax, apple wax, hardened fats, fatty acid esters, fatty acid glycerides, fatty acid triglycerides, polyethylene glycol waxes, mineral oils, isoparaffin oils, paraffin oils, squalane, sunflower seed oil, coconut oil, castor oil, lanolin oil, jojoba oil, corn oil and soybean oil.

17. A method of making a hair treatment gel, said method comprising the steps of:
a) making a gel mass with an aqueous solvent base; and subsequently
b) keeping the gel mass at temperature above room temperature until a gel-type solid is produced;
wherein the gel mass comprises at least one first polymer, which is a hydrocolloid based on a nonionic or anionic polysaccharide; at least one second polymer, which is a homopolymer or copolymer, which comprises at least one monomer selected from the group consisting of acrylamidoalkylsulfonic acids, salts of acrylamidoalkylsulfonic acids, methacrylamidoalkylsulfonic acids and salts of methacrylamidoalkylsulfonic acids; and at least one third polymer consisting of at least one amphiphilic polymer that is different from said at least one first polymer and at least one second polymer.

18. A hair styling gel made by the method according to claim 17.

19. A method of using the hair treatment composition of claim 1 for increasing volume of a hairstyle and/or for structuring a hairstyle.

20. A method of increasing volume of a hairstyle and/or structuring the hairstyle using the hairstyling gel made by the method of claim 17.
